(19) United States
(12) Patent Application Publication
(10) Pub. No.: US 2026/0130623 A1
(43) Pub. Date: May 14, 2026

(54) SYSTEM AND METHOD FOR CHARACTERIZING BONE MINERAL DENSITY IN PATIENTS UNDERGOING PRIMARY TOTAL KNEE ARTHROPLASTY

(71) Applicant: NEW YORK SOCIETY FOR THE RELIEF OF THE RUPTURED AND CRIPPLED, MAINTAINING THE HO, New York, NY (US)

(72) Inventors: Fernando Quevedo Gonzalez, Stamford, CT (US); Joseph Lipman, New York, NY (US); Timothy Wright, New York, NY (US); David Mayman, New York, NY (US); Jonathan Vigdorchik, New York, NY (US); Eytan Debbi, New York, NY (US); Cynthia Kahlenberg, New York, NY (US); Peter Sculco, New York, NY (US)

(73) Assignee: NEW YORK SOCIETY FOR THE RELIEF OF THE RUPTURED AND CRIPPLED, MAINTAINING THE HO, New York, NY (US)

(21) Appl. No.: 19/390,367

(22) Filed: Nov. 14, 2025

Related U.S. Application Data
(63) Continuation-in-part of application No. PCT/US24/29486, filed on May 15, 2024.
(60) Provisional application No. 63/466,482, filed on May 15, 2023.
(60) Provisional application No. 63/722,939, filed on Nov. 20, 2024.
(60) Provisional application No. 63/754,980, filed on Feb. 6, 2025.

(51) Int. Cl. A61B 5/00; A61B 5/055; A61B 6/00; A61B 6/03; A61B 6/12; A61B 6/46; A61B 6/50; A61B 8/00; A61B 8/08; G06T 7/00; G06T 11/00

(52) U.S. Cl. CPC A61B 5/4509; A61B 5/0035; A61B 5/055; A61B 5/4848; A61B 5/743; A61B 5/746; A61B 6/032; A61B 6/12; A61B 6/466; A61B 6/505; A61B 6/5217; A61B 6/5247; A61B 8/0833; A61B 8/0875; A61B 8/461; A61B 8/5223; A61B 8/5261; G06T 7/0016; G06T 11/00; G06T 2207/30008; G06T 2210/41

(57) ABSTRACT

An implant type or a process of implant fixation is automatically identified by processing surgical plan information. Further, bone mineral density information associated with a patient is determined by processing at least one image. The determined bone mineral density information and patient information representing at least demographics associated with the patient are processed, and a selection of a plurality of patients that previously underwent arthroplasty is made. Outcome information representing results of arthroplasty procedures is processed and a threshold is established representing a biomechanical tolerance. The bone mineral density information is processed to be determined associated with the patient is determined to be above or below the threshold.

### CROSS-REFERENCE TO RELATED APPLICATIONS

The present patent application is a continuation-in-part of International Patent Application No. PCT/US24/29486, filed May 15, 2024, which claims priority to U.S. Provisional Patent Application No. 63/466,482, filed May 15, 2023, and this application claims priority to and the benefit of U.S. Provisional Patent Application No. 63/754,980, filed Feb. 6, 2025 and U.S. Provisional Patent Application No. 63/722,939, filed Nov. 20, 2024, each of which is hereby incorporated by reference, as if expressly set forth in its respective entirety herein.

### FIELD

The present disclosure relates, generally, to data management and communications and, more particularly, to a system and method for analyzing bone mineral density to provide surgical recommendations for component choice, alignment, and fixation technique.

### BACKGROUND

Cementless total knee arthroplasty (TKA) has regained interest due to the potential for long-term biologic fixation. The use of cementless fixation in total knee arthroplasty has rapidly increased recently, from fewer than 5% of all primary TKA procedures performed in the United States in 2017 to over 20% in 2022. Such rapid adoption in cementless TKA is backed by substantial advancements in implant design. For example, arthroplasty implants include use of highly porous three-dimensional (3D) printed materials, as well as an incorporation of optimized keels and pegs. Further surgical technology (e.g., robotic assistance) has also improved cementless TKA, thereby providing improved conditions for initial fixation stability and rapid bone ingrowth.

For the same reasons as TKA, cementless fixation is also becoming increasingly popular in partial knee replacements, like unicompartmental knee replacements.

Generally, cementless fixation has been preferred for younger and active patients, who are thought to benefit from increased longevity of biologic fixation. Younger and active patients also are believed to have higher bone quality than older patients, which can further enhance the chance of achieving secure biologic fixation.

Unfortunately, characteristics of patients who are suitable candidates for cementless fixation remain unclear, which can result in a barrier to rational adoption of cementless fixation. Consequently, revisions in TKA may be required, for example, due to aseptic loosening, which is more common for the tibial component than the femoral component.

In order to avoid loosening in cementless fixation procedures, adequate bone support is needed to ensure stable initial fixation that can lead to bone ingrowth, as well as to avoid bone collapse under the loads that arise during daily activities.

It has been shown that joint loads and bone mineral density (“BMD”), an important marker of bone strength, can be impacted by patient characteristics and surgical choices. For example, patients with preoperative varus alignment have been shown to have denser bone under the medial half of the tibial plateau, while preoperative valgus alignment have been shown to have denser bone laterally. Additionally, varus/valgus alignment during surgery can impact the BMD of the bone directly supporting the implant and the joint loads.

The present system and method address these and other deficiencies in the art, and it is with respect to these and other considerations that the disclosure made herein is presented.

### SUMMARY OF THE INVENTION

By way of introduction and overview, a computerized method and system are provided herein. In one or more implementations of the present disclosure, at least one of an implant type and a process of implant fixation is identified by at least one computing device processing surgical plan information representing details associated with an arthroplasty procedure for a patient. The at least one computing device can determine by processing at least one image of the patient, bone mineral density information associated with a bone the patient. Further, the at least one computing device can select, by processing the determined bone mineral density information and patient information representing at least demographics associated with the patient, a plurality of patients that previously underwent arthroplasty including at least some of the details associated with the arthroplasty procedure for the patient. The at least one computing device can establish, by processing outcome information representing results of arthroplasty procedures respectively associated with the plurality of patients, a threshold representing a biomechanical tolerance of the implant fixation. Further, the at least one computing device can determine, by processing information associated with at least the determined bone mineral density information, that biomechanics of implant fixation associated with the arthroplasty procedure for the patient is above or below the established threshold. Information associated with the determination that the bone mineral density information associated with the patient is above or below the established threshold can be provided by the at least one computing device.

In one or more implementations of the present disclosure, the outcome information further represents arthroplasty procedures that succeeded and arthroplasty procedures that failed due to mechanical reasons.

In one or more implementations of the present disclosure, establishing the threshold further includes the at least one computing device processing implant and fixation information representing implants, fixation, implant migration, and pain associated with the arthroplasty procedures respectively associated with the plurality of patients.

In one or more implementations of the present disclosure, the at least one computing device can compare the determined bone mineral density information with information representing bone mineral densities of a plurality of patients that previously underwent knee arthroplasty.

In one or more implementations of the present disclosure, the automatically provided information is at least one of an alert displayed on a screen display, an audible alert, and a vibratory alert.

In one or more implementations of the present disclosure, the automatically provided information includes a recommendation for at least one of component choice, alignment, and fixation technique.

In one or more implementations of the present disclosure, the automatically provided information is instructional to configure a robotic surgical system.

In one or more implementations of the present disclosure, the at least one computing device can combine the determined bone mineral density information as input to a computational model with load information associated with biomechanics of the patient to provide the recommendation. In one or more implementations of the present disclosure, BMD information can be used to make the decision, as shown and described herein. Moreover, BMD information can be utilized in combination with loading information to investigate adequacy of fixation, such as shown and described herein. Still further, BMD information and loading information can be inputs to a computational model that determines the relative risk of bone failure based on load transfer between implant and bone.

In one or more implementations of the present disclosure, the at least one computing device can utilize the computed bone mineral density and generic loading information or the load information associated with biomechanics of the patient as input to the computational model to provide the recommendation.

In one or more implementations of the present disclosure, outcome of the model relates to an ability of the implant to achieve and maintain fixation following the arthroplasty for the patient.

In one or more implementations of the present disclosure, the outcome includes information associated with at least one of: implant motion relative to the bone; subsidence; bone strength; bone strains; bone stress; stress shielding; and interfacial stress.

In one or more implementations of the present disclosure, the outcome represents a likelihood of a failure of at least one of: a bond between components of the arthroplasty and the bone; the bone.

In one or more implementations of the present disclosure, the components of the orthopedic implant include at least one of: cement; an implant; a screw; a pin; and a plate.

In one or more implementations of the present disclosure, the at least one computing device can convert, by processing the at least one image of the patient, Hounsfield units to bone mineral density information.

In one or more implementations of the present disclosure, the at least one image of the patient is a computerized (CT) scan, magnetic resonance imaging (MRI), or a plurality of images with three-dimensional information.

Additional features, advantages, and embodiments of the disclosure may be set forth or apparent from consideration of the detailed description and drawings. It is to be understood that the foregoing summary of the disclosure and the following detailed description and drawings provide non-limiting examples that are intended to provide further explanation.

### BRIEF DESCRIPTION OF THE DRAWINGS

Aspects of the present disclosure will be more readily appreciated upon review of the detailed description of its various embodiments, described below, when taken in conjunction with the accompanying drawings, of which:

FIG. 1 is a block diagram illustrating an example implementation of the present disclosure and that represents a plurality of devices and the flow of information associated with the devices;

FIG. 2 is a block diagram that illustrates functional elements of one or more of a data processing apparatus or computing device;

FIG. 3 illustrates a process flow resulting from a standardized protocol used for CT scans and a BMD reference phantom placed in the field of view of each computerized tomography (CT) scan;

FIG. 4 illustrates decreasing BMD from proximal to distal, including the total cut, the medial half, and the lateral half above and below respective cuts;

FIG. 5 illustrates a process flow including a BMD analysis section, a bone-implant interaction analysis section, and a surgical procedure recommendation section, in accordance with an example implementation of the present disclosure;

FIG. 6 illustrates a process flow including a joint loading analysis section, a bone-implant interaction analysis section, and a surgical procedure recommendation section, in accordance with an example implementation of the present disclosure;

FIG. 7 illustrates a process flow including BMD analysis section and a joint loading analysis section, as well as a bone-implant interaction analysis section and a surgical procedure recommendation section, in accordance with an example implementation of the present disclosure;

FIG. 8 is a graph identifying dynamic KAM and static KAM in connection with joint loads;

FIG. 9 is a process flow illustrating steps associated with an example implementation of the present disclosure;

FIG. 10A illustrates a process of computing distribution of bone failure using a finite element model constructed from preoperative CT-scan, a pre-surgical plan, and implant geometry, in accordance with an example implementation of the present disclosure;

FIG. 10B illustrates example graphs representing risk of bone failure, in accordance with an example implementation of the present disclosure; and

FIG. 10C illustrates risk of failure, following cemented fixation or uncemented fixation, in connection with low bone mineral density and high bone mineral density, respectively, in accordance with an example implementation of the present disclosure.

### DETAILED DESCRIPTION OF CERTAIN IMPLEMENTATIONS

By way of introduction and overview, the present disclosure addresses how joint loads (e.g., knee joint loads) and bone density affect the ability of a patient's bone to resist static and cyclic loading and subsequent long-term implant fixation, including with regard to total knee arthroplasty (TKA). In one or more implementations of the present disclosure, information is provided that is associated with knee joint loads and the bone mineral density distribution of periarticular bone for patients who are expected to undergo or currently undergoing TKA. Systems and methods of the present disclosure operate to characterize bone mineral density (BMD), knee joint loads and moments, and the transfer of these loads between implant and bone, including for the proximal tibia. Nonetheless, the present disclosure can also be applied to partial knee replacement (e.g., unicompartmental knee replacement) or other bone and joint procedures for, for example, the distal femur, the hip, the ankle, or the elbow.

From a biomechanical standpoint, lower BMD has been associated with increased micromotion of tibial baseplates, which can result in formation of fibrous tissue instead of bone. As used herein, biomechanics relates, generally to how the structure and function of a biological system respond to the motions and loads acting upon the system.

Further, lower BMD is also associated with increased risk of bone failure underneath the tibial baseplate and subsequent implant migration. For example, patients with low BMD have experienced greater migration as measured through Radio Stereometric Analysis (RSA). Accordingly, an inverse relationship between implant migration and BMD has been shown. Traditionally, BMD is measured through Dual Energy X-ray Absorptiometry (DEXA), typically at central sites like the femoral neck or the spine. While relationships have been established between these central measures of BMD and BMD at the knee (e.g., the proximal tibia), and while DEXA can also be obtained at the knee (e.g., at the proximal tibia), DEXA does not provide information about the three-dimensional spatial distribution of BMD, which may be important for implant subsidence. In addition to BMD, higher joint forces and moments can relate to a greater risk of bone failure underneath the implant and higher implant micromotion. For example, patients with larger flexion and varus moments can be at increased risk of implant subsidence and high micromotion.

The present disclosure addresses concern of aseptic loosening, which remains a common cause of TKA revision, particularly as the use of cementless TKA continues to rise. For example, the present disclosure provides information that is usable to identify patients who are suitable candidates for biologic (cementless) fixation. In one or more implementations, patient bone mineral density of the proximal tibia and/or knee joint loads can be characterized in advance of TKA. BMD can be provided via analysis of imagery including, but not limited to three-dimensional distribution of computerized tomography (CT) scans of patients expected to undergo (or currently undergoing) TKA, including with respect to a location of the planned intraoperative tibial cut. Loads can be provided, for example, via analysis of ground reaction forces that are acquired and synchronized with radiographic images.

Although many of the examples shown and described herein regard radiographic images, it is to be appreciated that other imaging technologies are supported by the teachings herein. For example, magnetic resonance imaging (MRI), ultrasound, or other imaging can be supported in accordance with the teachings herein.

In previous known scenarios, information regarding bone mineral density is, generally, unavailable at the time of surgery and surgeons often decide which patients should receive cemented or cementless implants based on bone strength surrogates, like age or sex, or based on intraoperative assessment. The present disclosure recognizes, however, that sex or age are not suitable surrogates of bone mineral density. Accordingly, the present disclosure identifies an important overlap in bone mineral density among males and females across different age groups. For example, at the entire tibial cut, the medial half of the cut, or the lateral half of the cut, bone mineral densities are not influenced by age for men, while women ≥70 years can have significantly lower BMD than women between 60 and 70 years. However, despite the effect of age on bone mineral densities for younger women, differences in BMD between patients who received cemented or cementless fixation may not be significant. Paradoxically, women who receive cemented implants can have, potentially, denser bone than other women of similar age who received cementless implants.

Instead of the traditional surrogates for bone quality (e.g., sex, age), values of BMD can be analyzed in the context of prior patients who received a similar procedure, to determine whether BMD conforms to the norm or is higher/lower than prior patients, thereby helping the surgeon decide on the type of fixation (i.e., cemented or cementless).

Moreover, it is recognized herein that an analysis based exclusively on BMD does not account for the patient-specific joint loads and moments that can also contribute to implant failure. As such, a patient can generate joint forces and moments sufficiently large to cause implant failure, even if they have high BMD.

These knee joint forces and moments can be calculated from the ground reaction forces and their position relative to the knee. The traditional methodology involves a complex, costly, time-consuming, and highly specialized study on a dedicated motion analysis laboratory to determine the dynamic joint forces and moments during daily activities (e.g., walking on level ground). However, static surrogates that represent the most critical case from daily activities can also be obtained. To this end, ground reaction force measurements obtained with a force plate can be combined with synchronized radiographs, allowing to determine the position of the knee center relative to the ground reaction forces.

More particularly, biplane radiographs can be utilized to obtain the three-dimensional spatial position of the knee joint center relative to a reference coordinate frame of the force plate. These acquisitions can be performed during different poses, e.g., bipedal stance and single leg stance, for a comprehensive assessment of the knee loading environment. As such, the two-dimensional projection of any three-dimensional object can be modeled with a pinhole camera model, where an extrinsic transformation expresses the relationship between a global reference frame and the camera reference frame, while an intrinsic camera transformation matrix generates the two-dimensional projection of the object in the image reference frame. The extrinsic and intrinsic transformation matrices are a function of the geometry of the system, including the distance between the camera and the image detector or the pixel dimensions in mm. When a biplane system is utilized, a unique relationship exists between the two-dimensional projections of the object and its three-dimensional position. This relationship depends on the relative position of the two cameras. Therefore, by knowing the relative positions of the cameras and detectors within the system and some image parameters, like the size of each image pixel in mm, one can establish a unique set of extrinsic and intrinsic matrices that allow determining the three-dimensional position of an object from its biplane image representations. The intrinsic and extrinsic matrices can be obtained through a calibration process, where a known three-dimensional object is imaged multiple times with the system and the various parameters are optimized.

Systems and methods set forth in the present disclosure can be implemented at least in part with an EOS® biplane radiographic system. EOS® systems, generally, include a fixed geometry of the system in which the images are created with a fan-beam projection such that no vertical magnification exists. Further, the system corrects for any horizontal magnification, producing images without magnification. In this case, the pinhole camera model must be modified to not include vertical magnification and to account for the correction introduced by the system. Such corrections can be incorporated into the intrinsic camera transformation matrix and can be determined by knowing the geometry of the system or by a calibration step. For example, a magnification correction can be implemented so that the image is of true magnitude (i.e., true size) at the patient's center. Therefore, the image can be corrected by the ratio of the distances from the source to the patient and from the source to the detector. The three-dimensional position of any object can be obtained by solving the inverse projection problem for both cameras simultaneously. As such, the three-dimensional position of the knee center can be obtained by selecting corresponding points on the frontal (anterior-posterior) and lateral radiographs and solving the inverse projection problem. Similarly, the position of known points in the force plates can be obtained. These points can correspond, for example, to points along an edge of the force plate, allowing the definition of the plate's orientation and position relative to the EOS® image system. Consequently, the radiographic measurements of the knee joint center can be spatially and temporarily synchronized with the force place measurements, allowing to calculate the joint moments as the product of the ground reaction forces and their distances to the knee joint center.

The use of EOS® and other image systems herein are submitted to be implementation examples, and one of ordinary skill will recognize that the teachings herein can include other single plane or biplane radiographic systems.

The present disclosure recognizes that these static loads may be adequate surrogates of dynamic joint loading. In particular, the knee adduction moment during single leg stance is a surrogate of the peak dynamic knee adduction moment during walking on level floor.

These loads and moments along with the BMD information can be further utilized as inputs to computational models (e.g., finite element models) to provide estimates of the load transfer between implant and bone and determine the combined effects of BMD and joint loading on the risk of implant subsidence, bone-implant micromotion, or bone strains, for example.

Accordingly, the present disclosure addresses the determination whether to recommend a respective type of fixation by providing a reduced emphasis on sex and age, at least in isolation, as potentially limited surrogates of bone quality. Such recommendation can be formatted in various ways, including as a textual notification, an audible notification, a vibratory notification, as a robotic surgical plan, or other suitable format. The present disclosure applies a more detailed analysis of the bone mineral density distribution and/or the joint loads and moments with the possibility of considering bone mineral density and joint loads and moments as inputs to biomechanical computational (e.g., finite element) models to analyze the load transfer between implant and bone with the ultimate goal of determining which patients may be well-suited as candidates for cementless TKA and, conversely, which patients may require cemented fixation.

Moreover, the present disclosure allows for analyzing the BMD, joint forces and moments, or bone-implant interaction in the context of normative values to establish whether the values of these variables or a given patient are high or low compared to other patients who previously received the same operation. For example, normative curves of BMD along the most proximal 12 mm of the tibia on prior patients against which a patient's BMD can be plotted to analyze their bone mineral density relative to a cohort of prior patients who received TKA. Similarly, the normative values of knee joint forces and moments before surgery, after surgery, or their changes can be obtained from prior patients and utilized to define normative values to analyze the knee joint loads (i.e., forces and moments) of a patient relative to a cohort of prior patients who received TKA.

Further, one or more computing devices can be configured to limit one or more factors that can result in inconsistency by analyzing and factoring respective implants and implant designs, for example, restricting the analysis to patients who received a cruciate-retaining insert. Even further, clinically relevant thresholds can be established for determining bone mineral density by comparing bone mineral density of successful procedures and procedures that failed for mechanically related reasons, or by relating BMD, joint loads and moments, or the interaction between implant and bone to outcomes such as implant migration, pain, or patient-reported outcomes. Various aspects of bone quality, such as bone turnover or collagen content, can be additionally factored by one or more computing devices to account for assessing bone quality as a multifactorial metric, including beyond bone mineral density which may only partially capture the ability of the bone to resist the loads imparted to the joint during daily activities. Still further, the present disclosure can include analyzing temporal changes in knee joint loads and moments that can occur after TKA and the associated changes in load transfer, which can affect the ability of the bone to resist loads. Accordingly, an evaluation of postoperative changes in knee joint loads and moments and the associated changes in load transfer can be further included in one or more respective implementations of the present disclosure.

Still further, one or more computing devices that are configured by executing instructions stored on non-transitory processor readable media can determine information associated with affected bone mineral density. For example, patients undergoing robotic-assisted TKA have pre-operative computed tomography scans. The Hounsfield units of the computed tomography scans can be converted to BMD with the use, for example, of a calibration phantom. Calibration can be synchronous when the phantom is included in the actual scan or asynchronous if the phantom is scanned in the same machine, with the same scanning parameters, but at a different time point to the scan of interest. Phantomless calibration techniques that utilize reference BMD values for specific tissues and air can also be utilized in this step. Using the robotic surgical plan, the one or more computing devices determine (e.g., using information provided on a planned surgical procedure) the planned tibial cut and compute the BMD distribution, for example, of 1 mm spaced cross-sections parallel to the cut, from 2 mm above the cut to 10 mm below. Using the information therefrom, bone mineral density can be analyzed with respect to various patient factors, including sex, age, alignment, and type of fixation. The distribution of BMD can be further analyzed in the context of prior patients who underwent TKA with a similar implant. The BMD distribution can also be computed for various possible planned positions of the implant to help decide the alignment and fixation method of the implant for each patient.

The present disclosure provides an ability to determine, by one or more computing devices that are configured by executing instructions stored on non-transitory processor readable media, information associated with the affected joint forces and moments. For example, patients undergoing TKA may receive biplanar radiographs, for example with the EOS® biplanar radiographic system. These can be synchronized with force plate measurements of the ground reaction force. A static equilibrium problem can be solved to determine the joint moments as the product of the ground reaction forces and their distance to the knee center and the joint forces as the external forces plus those generated by the muscles. This analysis can be done preoperatively and the expected joint forces and moments after surgery can be predicted by finding similar patients with similar preoperative joint forces and moments and desired alignment.

The present disclosure provides an ability to determine, by one or more computing devices that are configured by executing instructions stored on non-transitory processor readable media, information associated with the bone-implant interaction. For example, using the BMD distribution and the computed joint forces and moments, patient-specific computational finite element models can be generated to compute the relative motion between the implant and the bone, which is a marker for bone ingrowth, or the bone stresses and strains, which can be related to the strength of the bone to determine the risk of bone failure. The relative motion between implant and bone can be compared to known thresholds for ingrowth. For example, it has been shown that above 150 micrometers of relative motion, only fibrous tissue is created between implant and bone, while below 40 micrometers bone ingrowth can occur. Similarly, the bone strains can be compared to known strength of the bone. Also, the failure compressive strain of the tibia bone has been established in 7,300 microstrain, and the failure tensile strain, 6,500 microstrain. By comparing the bone strains to these established thresholds, for example, by expressing the strain as a percent of the bone strength, the amount of bone at risk of mechanical failure can be computed and reported. Therefore, the surface area of the implant amendable to ingrowth can be computed as the area where micromotions and strains are within acceptable limits and reported. Additionally, the bone at risk of failure or the micromotion can be related to, for example, the postoperative motion of the implant relative to the bone or implant subsidence, to determine an acceptable threshold for these variables. Such threshold may be related to mechanical failure of the fixation or to an unacceptable motion of the implant because, for example, it increases the joint gaps and laxity. The recommendation of using cemented or cementless fixation or the recommendation of a particular implant position and size, therefore, can be done based on this information.

In one or more implementations of the present disclosure, inputs to a model can be used for assessing the load transfer pathway for the implant and/or bone of a respective patient. For example, two patients having similar bone mineral density can have different risk of failure. Even where the patients have the same distribution of density, they may experience different risk of failure if they load their joints differently. The bone mineral density distribution can be slightly different, meaning for example, that one patient has higher density near or at the front while one patient has higher density near or at the back, resulting in different load transfer patterns that create, for example, differences in strains in the bone, leading to differences in degrees of risk. Bone density distribution can, therefore, affect how loads are transferred in conjunction with the loads themselves and, therefore, result in different risks of failure.

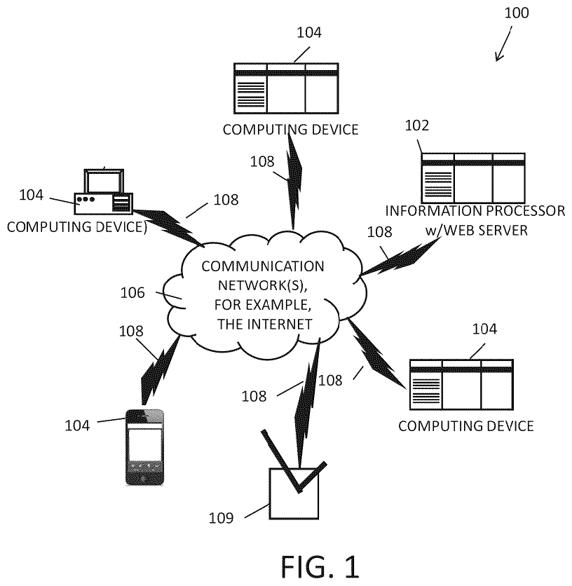

Referring now to FIG. 1, a block diagram is shown illustrating an example implementation of the present disclosure and that represents an association of a plurality of devices and the flow 108 of information associated with the devices. In the example shown in FIG. 1, various computing devices 102 and 104 are shown, each capable of executing desktop and/or mobile computing device web browser application(s) including MICROSOFT EDGE, INTERNET EXPLORER, CHROME, FIREFOX, and other (e.g., SAFARI, OPERA). In addition to standard web browser application functionality, user information can be gathered via Push Notifications, and information can be retrieved from a computing device using a “REST” interface. Various mobile devices running different operating systems are shown, including IOS, ANDROID and other (e.g., PALM, WINDOWS or other mobile device) operating system.

In the example shown in FIG. 1, one or more data processing apparatuses 102 is operatively coupled to one or more user computing device(s) 104. Devices 102/104 can be respectively operated by one or more users skilled in the use of the proposed workflow, including, but not limited to, healthcare providers and associated staff, medical specialists, and/or biomechanical specialists. Healthcare providers can include, for example, physicians, physician assistants, nurses, therapists and/or other providers of healthcare services. Biomechanical specialists can include, for example, engineers specialized in biomechanics. Data processing apparatus 102 and/or user computing device 104 can be operable to access and/or store various information on database(s) 103 including, for example, historic medical and procedure information patients, physicians, devices, or the like. Also illustrated in FIG. 1 is robotic surgery system 109 which, in various forms as known in the art can include, for example, one or more processors, networking interface, imaging technology, mechanical arms, and surgical instruments.

Continuing with reference to FIG. 1, network 106 is illustrated, which can be configured as a local area network (LAN), wide area network (WAN), Peer-to-Peer network (“P2P”), multi-Peer network, the Internet, one or more telephony networks or a combination thereof, that is operable to connect data processing apparatus 102 and/or devices. Though many of the examples and implementations shown and described herein relate to product and/or service recommendations, many other forms of content can be provided and/or delivered by system 100.

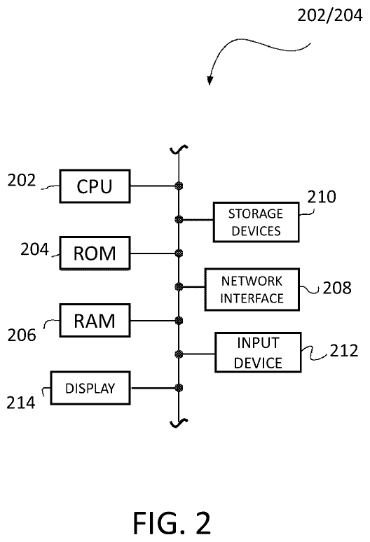

FIG. 2 is a block diagram that illustrates functional elements of one or more of data processing apparatus 102 or computing device 104 and preferably include one or more central processing units (CPU) 202 used to execute software code in order to control operations, including of data processing apparatus 102, read only memory (ROM) 204, random access memory (RAM) 206, one or more network interfaces 208 to transmit and receive data to and from other computing devices across a communication network, storage devices 210 such as a hard disk drive, solid state drive, universal serial bus (USB) drive, floppy disk drive, tape drive, CD-ROM or DVD drive for storing program code, databases and application code, one or more input devices 212 such as a keyboard, mouse, track ball and the like, and a display 214.

The various components of devices 102 and/or 104 need not be physically contained within the same chassis or even located in a single location. For example, storage device 210 can be located at a site which is remote from the remaining elements of computing devices 102 and/or 104 and can even be connected to CPU 202 across communication network 106 via network interface 208.

The functional elements shown in FIG. 2 (designated by reference numbers 202-214) are preferably of the same categories of functional elements preferably present in computing device 102 and/or 104. However, not all elements need be present, for example, storage devices in the case of mobile computing devices (e.g., smartphones), and the capacities of the various elements are arranged to accommodate expected user demand. For example, CPU 202 in computing device 104 can be of a smaller capacity than CPU 202 as present in data processing apparatus 102. Similarly, it is likely that data processing apparatus 102 will include storage devices 210 of a much higher capacity than storage devices 210 present in computing device 104. Of course, one of ordinary skill in the art will understand that the capacities of the functional elements can be adjusted as needed. For example, one or more graphics processing units (GPU) can be utilized for processing and providing functionality shown and described herein. In addition, or in the alternative, a cluster of computing devices can work to provide functionality shown and described herein.

The nature of the present disclosure is such that one skilled in the art of writing computer executed code (software) can implement the described functions using one or more or a combination of a popular computer programming language including but not limited to C++, JAVA, ACTIVEX, HTML, XML, ASP, SOAP, IOS, OBJECTIVE C, ANDROID, TORR, PYTHON, MATLAB, and various web application development environments.

As used herein, references to displaying data on computing device 104 refer to the process of communicating data to the computing device 104 across communication network 106 and processing the data such that the data can be viewed on the user computing device 104 display 214 using a web browser, custom application or the like. The display screens on computing devices 102/104 present areas within system 100 such that a user can proceed from area to area within the system 100 by selecting a desired link. Therefore, each user's experience with system 100 will be based on the order with which (s)he progresses through the display screens. In other words, because the system is not completely hierarchical in its arrangement of display screens, users can proceed from area to area without the need to “backtrack” through a series of display screens. For that reason and unless stated otherwise, the following discussion is not intended to represent any sequential operation steps, but rather the discussion of the components of system 100.

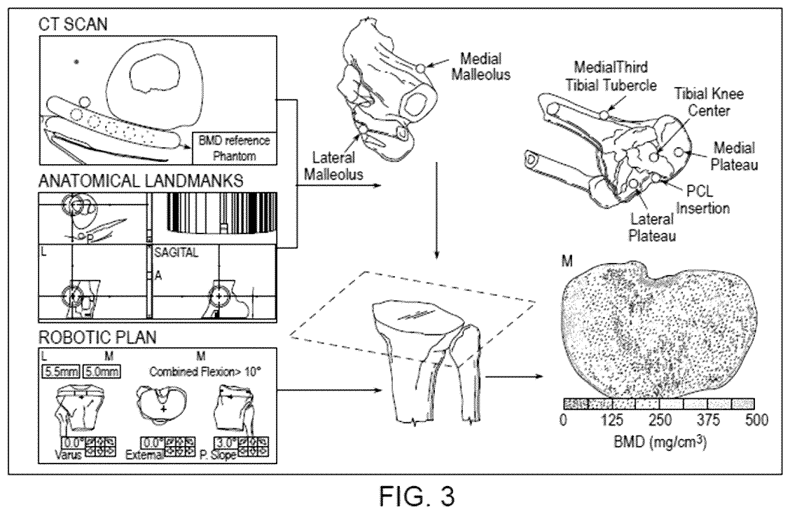

One or more computing devices can be configured to process information associated with patients who received a pre-operative computed tomography (CT) scan, prior to robotically assisted primary TKA. For example, information is processed resulting from a standardized protocol used for CT scans (e.g., 120 kV, 200 mA, slice spacing: 0.625 mm) and a BMD reference phantom placed in the field of view of each CT scan. (See, for example, FIG. 3). As illustrated in FIG. 3, the BMD reference phantom can be configured with five rods, each with a different known quantity of K₂HPO₄ and water, such that Hounsfield Units can be converted to volumetric BMD, which is highly correlated with physically measured bone ash density.

Each of the patients can receive the same implant design with posterior cruciate ligament (PCL) retention. Other types of implants can also be considered. Using the information identified therewith, one or more computing devices can preoperatively determine and analyze the bone density of knee arthroplasty patients through the CT and calculate respective loading at the knee through EOS® images synchronized with force plate measurements to provide surgical recommendations for component choice, alignment, and fixation technique.

More specifically, tibiae can be segmented from the CT scans, and the Hounsfield Units can be converted to units of BMD (mg/cm³ of K₂HPO₄) on a voxel-by-voxel basis, using the patient- and scan-specific relationships obtained with the BMD reference phantoms. To this end, a scan-specific linear relationship can be obtained between Hounsfield Units and BMD by relating the Hounsfield Units of materials with known BMD (e.g., a BMD calibration phantom), to their known BMD values. Such relationship can be applied to each voxel to convert the Hounsfield Units to BMD. This provides a volumetric distribution of BMD for each patient, which can be analyzed relative to the superior-inferior, medial-lateral, and anterior-posterior axes of each tibia, defined from the bony landmarks utilized in creating the robotic surgical plan. These can be identified, for example, via image recognition processes, machine learning, and artificial intelligence, for matching their location on the CT-scans (FIG. 3). The tibial cut plane can be reproduced by considering the posterior slope (i.e., rotation around the medial-lateral axis), the varus-valgus alignment (i.e., rotation around the anterior-posterior axis), and the cut thickness relative to the reference landmarks on the plateaus, retrieved from the saved intraoperative robotic plan.

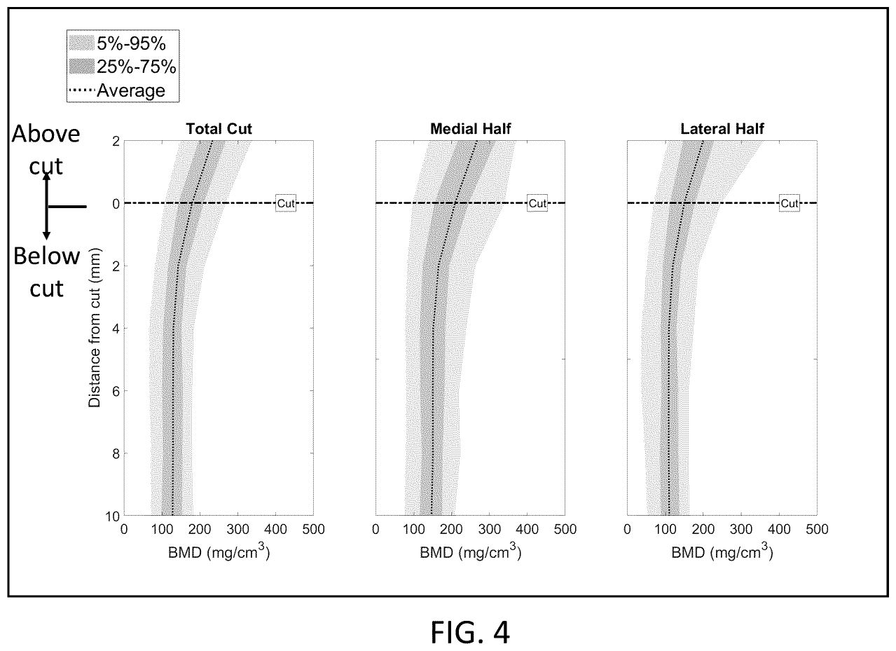

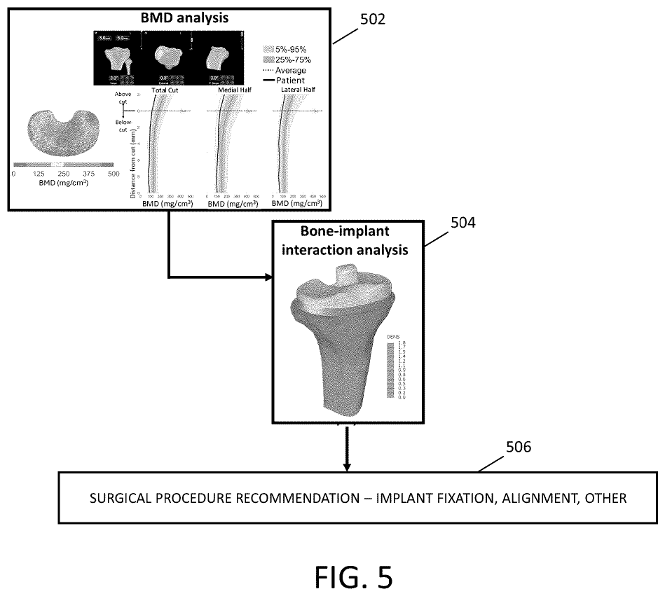

Continuing with the example illustrated in FIG. 3, from the volumetric BMD distribution, a two-dimensional distribution of BMD can be extracted at various cross-sections parallel to the robotic tibial cut. These cross sections can be equally spaced, for example, every 1 mm, from 2 mm above the cut to 10 mm below the cut. To this end, at each plane parallel to the tibial cut, a grid of pixels can be defined with the same dimensions as the voxel size in the axial slices (0.488×0.488 mm). Thereafter, one or more computing devices can be configured by executing instructions stored on non-transitory processor readable media to determine the intersection between each three-dimensional voxel and the different planes, resulting in the BMD distribution at each pixel in the plane. The average BMD can be calculated for the entirety of each cross-section, for the medial and lateral halves of the cross-section, or for any other subdivision of the cross-section. See, for example, FIG. 4. As illustrated in FIG. 4, percentages of patients having respective BMD (mg/cm³) are shown, including from 5% to 95% and from 25% to 75%. The computing devices can further analyze BMD as a function of tibial cut depth (i.e., distance from the tibial cut) and related the BMD at the robotic cut and the medial and lateral halves of the cut to the patient's sex, age, preoperative alignment (i.e., neutral/varus vs valgus), and to the method of fixation chosen intraoperatively (i.e., cemented vs cementless). The BMD analysis can be restricted to any region of the cut. For example, a baseplate can be virtually positioned onto the planned cut, according to a robotic surgical plan. Positioning of the baseplate can be done manually or automatically. Then, the BMD analysis can be restricted to the bone underneath the baseplate (see FIG. 5).

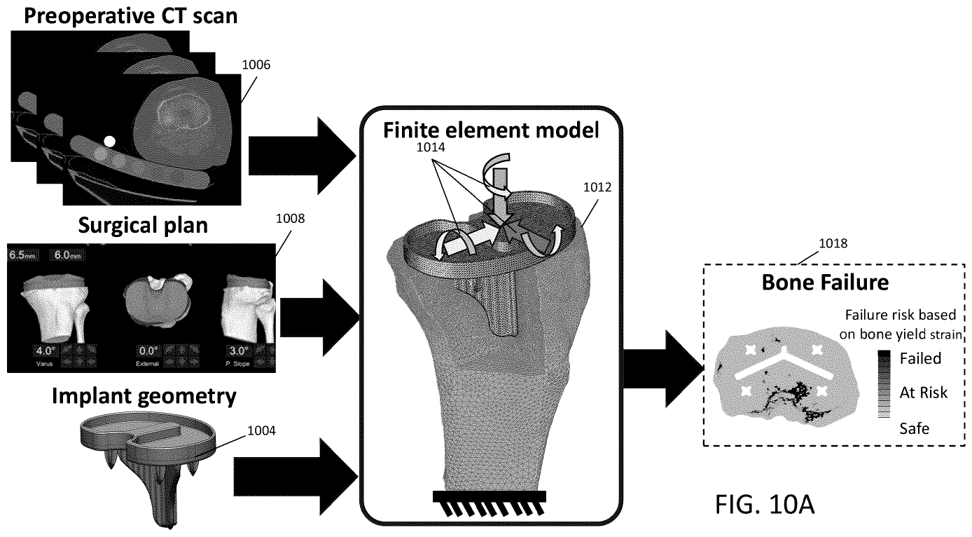

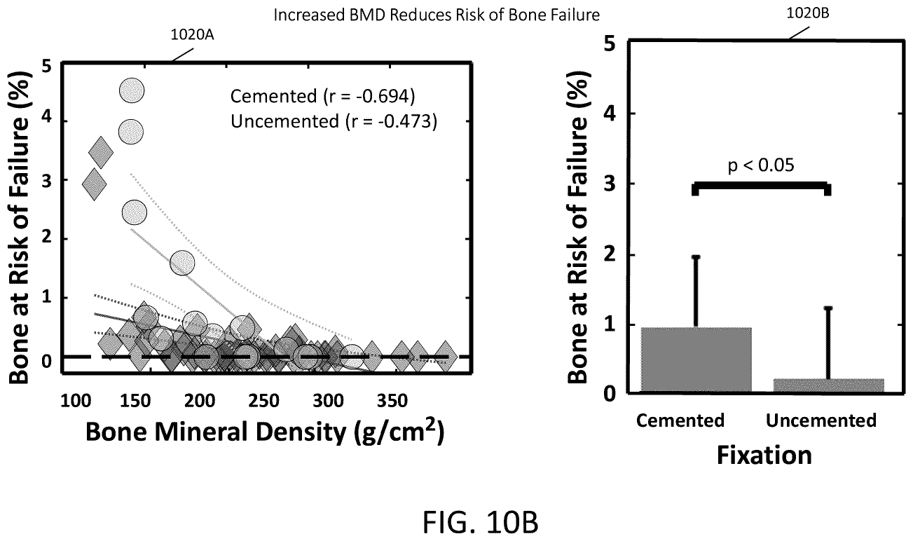

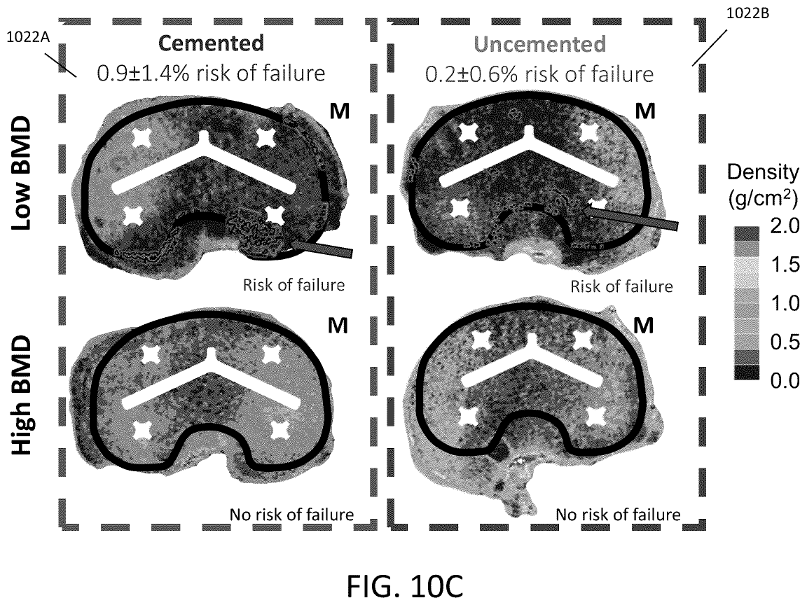

FIG. 5 illustrates a process flow including BMD analysis section, bone-implant interaction analysis section, and a surgical procedure recommendation section, in accordance with an example implementation of the present disclosure. As shown in FIG. 5, BMD analysis section 502 includes robotic plan information, and BMD distribution (see, for example, FIG. 3). Also included in BMD analysis section 502 is a calculated average BMD of each cross-section and for the medial and lateral halves of the cross-section (see, for example, FIG. 4), as well as a line representing the current patient's BMD. Thereafter, at Bone-implant interaction analysis section 504, a determination is made regarding the planned arthroplasty procedure and, thereafter, recommendations can be provided regarding implant fixation technique, alignment, or other recommendation, via surgical procedure recommendation section 506. A surgical recommendation can also be made based on the BMD values relative to the cohort of patients. For example, if the patient's BMD is in the lower 25% of the reference cohort, a warning message can inform of the relative low BMD of the patient and a suggestion can be made to consider a different method of fixation, for example cemented fixation, or to consider an alternative alignment. Furthermore, the bone volume segmented from the CT-scan can be utilized to generate a computational biomechanical model, for example, a finite element model or any other type of model that allows calculating the deformation of the bone under load. These loads can correspond to loads from daily activities or other loads. Loads can be calculated based on EOS® standing data. For example, by incorporating a force plate into the EOS, the ground reaction forces can be determined and related to the joint positions visible in the radiographs, thereby allowing to determine the joint loads, for example through a free body diagram. Moreover, a simplified loading regime based on the patient's weight and implant alignment can be factored and used as inputs, such as in connection with included. The simplified loads can be included as inputs, such as in connection with reduced risk of bone failure following arthroplasty as a function of BMD, including in connection with cemented and uncemented fixation (see, for example, FIG. 10B). The BMD distribution for the entire bone volume or a subregion of the bone volume (e.g., the most proximal 100 mm) can be used as input to the model to account for the non-homogeneous spatial distribution of bone material properties. To this end, the BMD can be converted to bone's elastic modulus utilizing empirical relationships. Furthermore, a virtual surgery can be performed according to the plan, to determine the bone-implant interaction, for example micromotion or bone strains, and utilize this information to further inform the surgical plan. The recommendation can then be made based on the computed values from the bone-implant interaction. Even further, the computational model can be incorporated into an algorithm to determine the bone-implant interaction for various alternative alignments or fixations of the implant to provide a surgical recommendation based, for example on the implant position that minimizes micromotion or bone strains. In one or more implementations of the present disclosure, one or more computing devices can be configured, for example, via an algorithm, to find the implant position that rests on the densest bone possible. Moreover, a recommendation can be generated and transmitted to perform a thicker cut and compensate with a thicker insert to reach a location of higher density (i.e., higher bone support). In one or more implementations, strains can require one taking into account loading through a simulation. The present disclosure includes use of information relating to BMD, including the generate and transmit a recommendation based on finding a position for an implant that has a maximum (or near maximum) BMD under the baseplate. Such recommendation can include moving the implant position so to support the implant resting on bone that has a higher-than-average BMD, thereby changing the position to a location that minimizes bone strain.

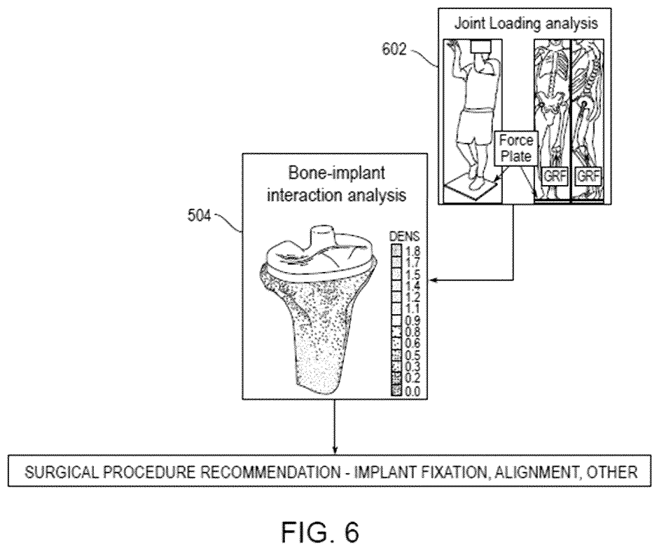

FIG. 6 illustrates a process flow including joint loading analysis section 602, which includes loading information (see, for example, FIG. 10A). Thereafter, at Bone-implant interaction analysis section 504, a determination is made regarding the planned arthroplasty procedure and, thereafter, recommendations can be provided regarding implant fixation technique, alignment, or other recommendation, via surgical procedure recommendation section 506. In addition to and similarly to the BMD analysis, an analysis can be performed based on the determined joint loads, such that a recommendation can be made for patients with loads that deviate from the normative values of a reference cohort of patients. Such recommendation can include using cemented fixation or changing the alignment if the loads are, for example, on the top 75% of the reference cohort of patients. The loading information can also be incorporated into an algorithm to determine the range of acceptable alignments, for example, as those for which the expected joint loads will be lower than the 75th percentile of the loads for the reference cohort of patients. Even further, the loading information can be also input into the computational model, for example, finite element model, to allow determining the bone-implant interaction under the patient-specific loading conditions.

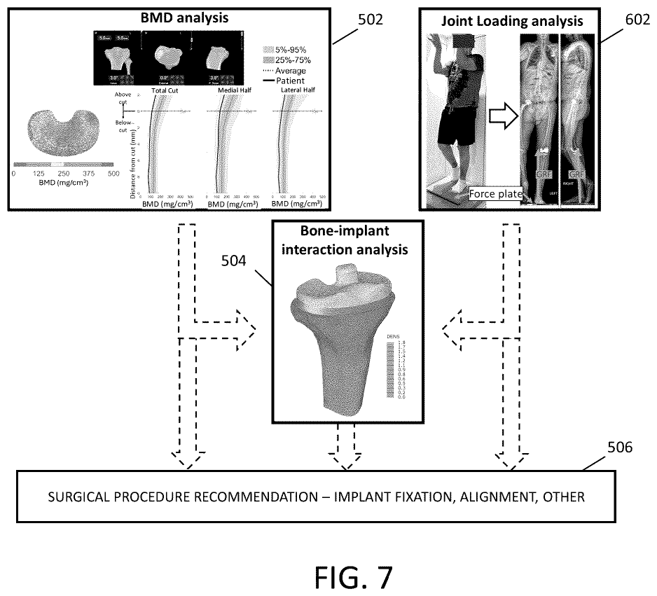

FIG. 7 illustrates a process flow associated with FIG. 7 analyzing BMD information or a combination of BMD information and loading information, as well as for using BMD information or a combination of BMD information and loading information as one or more inputs to a finite element model. As illustrated in FIG. 7, BMD analysis section 502 (FIG. 5) and joint loading analysis section 602 (FIG. 6), are shown, as well as bone-implant interaction analysis section 504. These are usable in connection with a planned arthroplasty procedure and, thereafter, recommendations can be provided regarding implant fixation technique, alignment, or other recommendation, via surgical procedure recommendation section 506. The information about joint loading can also be combined with the BMD information to provide a holistic and combined assessment for the purpose of preoperative planning. For example, the information about BMD can be analyzed jointly with the loading information in the context of a reference cohort of patients to determine whether the combined effect of BMD and joint loads is suitable for cementless fixation or may require alterations to the surgical plan, like an alternative fixation method or an alternative alignment. More particularly, patients with high BMD can generate large loads, which can jeopardize their fixation. Thus, a combined analysis can identify such situations and provide a recommendation. The recommendation can be further based into computational models that use as inputs the BMD and joint loads of the patient to determine the bone-implant interaction.

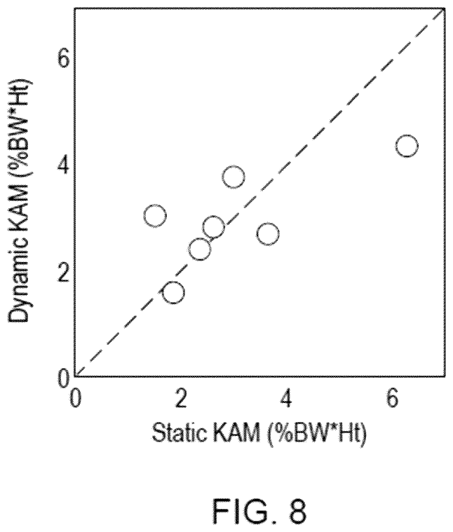

FIG. 8 is a graph identifying dynamic knee adduction moment (“KAM”) and static KAM in connection with joint loading. Dynamic knee adduction moment, as used herein, refers, generally to a measure of the force pushing the knee inward, or towards the midline of the body, during activities like walking. Static knee adduction moment refers, generally, to a calculated force that represents the bending moment in the frontal (coronal) plane of the knee, where the force is directed inward towards the body's midline. A static KAM can be used as either an input during a finite element analysis (e.g., via a finite element model) or used as an independent assessment of risk of implant failure, as discussed herein. As represented in FIG. 8, each dot in the graph corresponds to a different patient, demonstrating that the static Knee Adduction Moment obtained radiographically, on the horizontal axis, is an adequate surrogate marker of the dynamic knee adduction moment obtained during motion analysis, represented in the vertical axis, as evidenced by values close to the grey line that indicates equal values of static and dynamic knee adduction moments. Therefore, the static KAM can be used as a surrogate for dynamic KAM when loading the knee in the bone-implant interaction analysis.

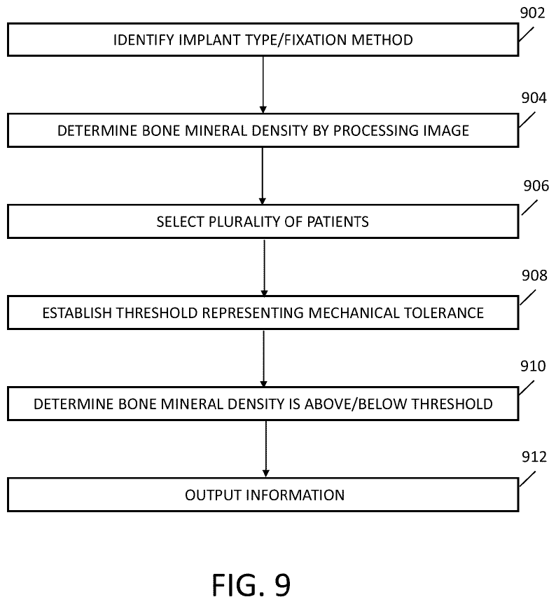

FIG. 9 is a process flow illustrating steps associated with an example implementation of the present disclosure. As illustrated in FIG. 9, at least one of an implant type and a process of implant fixation is automatically identified, by at least one computing device processing surgical plan information representing details associated with a knee arthroplasty procedure for a patient (step 902). Further, the at least one computing device determines, by processing at least one image of the patient, bone mineral density information associated with the patient (step 904). The at least one computing device selects, by processing the determined bone mineral density information and by the at least one computing device processing patient information representing at least demographics associated with the patient, a plurality of patients that previously underwent knee arthroplasty including at least some of the details associated with the knee arthroplasty procedure for the patient (step 906). Moreover, the at least one computing device establishes by processing outcome information representing results of arthroplasty procedures respectively associated with the plurality of patients, a threshold representing a mechanical tolerance of the implant fixation (step 908). In one or more implementations, the threshold value can represent a mechanical variable (e.g., micromotion, bone strains, bone mineral density). Such threshold can be determined by relating patient outcome information to a given patient's clinical history. Such outcome information can include, for example, implant failure, patients'levels of satisfaction, degrees of pain, or the like. Moreover, in one or more implementations, a micromotion threshold can be defined where micromotion exceeds a certain level. Furthermore, in one or more implementations, mechanical threshold can represent a percentage of % bone at risk of failure. If the percentage exceeds a certain value (e.g., exceeding a threshold), then one or more computing devices can be configured to generate and transmit an alert, recommendation, or other output directing an implant to be repositioned, cement be used, or other suitable information.

Accordingly, in one or more implementations of the present disclosure, the at least one computing device determines, by processing at least one instruction, the determined bone mineral density information associated with the patient is above or below the established threshold (step 910). A respective BMD threshold can be determined by examining the BMD of a large population of patients, locate cases associated with failure and determine those patients'respective BMD. In one or more implementations of the present disclosure, a recommendation, alert, or other output can be generated and transmitted in cases where a patient with BMD below the threshold, is at risk of failure, and is a candidate for receiving cemented implants. The information associated with the determination that the bone mineral density information associated with the patient is above or below the established threshold is provided by the at least one computing device (step 912).

Thus, as shown and described herein, the present disclosure provides a computational framework capable of providing a holistic understanding of knee biomechanics after total knee arthroplasty. The framework provides an objective evaluation of potentially important tradeoffs between the joint level mechanics and fixation level mechanics. The framework can be utilized then to optimize implant position to maximize longevity and function of total knee replacements and generate a patient-specific presurgical plan.

Further, in one or more implementations the proposed framework can be applied to generating pre-surgical plans for primary and revision joint arthroplasties.

Also, and as noted herein, known biomechanical studies provide detailed information regarding either the joint level mechanics or the fixation level mechanics.

FIGS. 10A, 10B, and 10C illustrate correlation of low bone mineral density to an increased risk of bone failure under TKA tibial implants, in accordance with an example implementation of the present disclosure. FIG. 10A illustrates an example process of computing the distribution of bone failure using a finite element model constructed from preoperative CT-scan, a pre-surgical plan, and implant geometry. The process includes creating patient-specific finite element models for evaluation of risk of bone failure, for example, under the baseplate, and relating the assessed risk to respective bone mineral density. Fixation of the tibial implant can be considered crucial toward longevity of total knee arthroplasty. It is recognized herein that failure of fixation represents a lead cause for TKA revision, and often involves varus subsidence of the tibial baseplate, likely due to bone failure under loads. While bone mineral density is a marker of bone strength, there is little differentiation between patients with cemented and uncemented implants. Thus, the choice of fixation based on the bone density alone, for example, cemented in cases where bone quality seems poor and uncemented in cases where bone quality seems adequate, may be insufficiently made. The present disclosure provides surgeons with aid during a decision-making process of a respective choice of fixation method outside of subjective measures of quality by incorporating loading information to determine the risk of bone failure.

The present disclosure further can include finite element models to bridge knowledge gaps, for example by comparing BMD distributions to areas of bone at risk of failure, by comparing load transfer differences between fixation methods, and by establishing thresholds for patients who may benefit from uncemented fixation. For example, FE models can be used for: comparing BMD distributions to areas of bone at risk of failure; comparing load transfer differences between fixation methods; and establishing thresholds for patients who may benefit from uncemented fixation. In operation, FE models can be used to compare (a) how BMD relates to the risk of bone failure and (b) the difference in risk of failure between patients who received cemented and uncemented TKA procedures.

With reference to FIG. 10A, a process is shown that includes Finite Element Analysis and Computer-Aided Engineering for modeling, analyzing, and simulating physical components (e.g., implants) in connection with arthroplasty. Model inputs can include a preoperative CT scan of a patient 1006 and information about a surgical plan 1008, patient-specific size implant geometries, and loading information. With a CT scan, for example, 3D sizing information can be made available, which can be referenced to the technique guides with the implant sizes. In one or more implementations of the present disclosure, a database can be referenced that stores 3D sizes of implants. In operation, a patient specific size can be determined, for example, based on a CT scan, bony landmarks, and a respective implant size. Thereafter, a determination can be made regarding what fits, for example, in the anterior/posterior (“AP”) and medial/lateral (“ML”) directions.

Implant models suitable for finite element modeling can be obtained, for example, from an input three-dimensional scan of physical parts. The scan can be converted to a volumetric solid body or used as a guideline to determine the appropriate dimensions and computer-aided design operations to generate the implant's computational model. In another example, a library of computer aided design (“CAD”) models can be stored for various sizes of implants. In one or more implementations of the present disclosure, a respective implant geometry can be selected from the library of models, in addition to or opposed to scanning geometry.

At least one of an implant type and a process of implant fixation (e.g., cemented or uncemented) can be identified by at least one computing device processing surgical plan information representing details associated with an arthroplasty procedure. Bone geometry and bone mineral density information associated with the patient can be determined and processed from the preoperative CT scan and can be utilized, along with the implant information, to create the finite element model 1012, in accordance with a surgical plan. The model can be simulated under appropriate loading scenarios 1014, can be utilized, along with the implant information, to create the finite element model 1012, according to a surgical plan. The model can be simulated under appropriate loading scenarios 1014, for example representing a demanding daily activity and to process outcome information representing the relative risk of bone failure, for example, the bone exceeding a specific strength threshold. The results of the model can be related to those of a plurality of patients to determine a threshold representing a biomechanical tolerance of implant fixation. Biomechanics of implant fixation associated with the arthroplasty procedure for the patient can be determined to be above or below the established threshold. The model can be utilized to evaluate alterations of the surgical plan (e.g., thinner or thicker resections, different angles of resection (varus/valgus), different rotations, bone properties, loading, or other relevant variables for the biomechanics of implant fixation. Effectively, implant geometry (top left) and a CT scan of the patient (bottom left) are inputs. Loads, boundary conditions and implant position (center) are included, and outputs are on the right, including bone that is at risk of failure.

Continuing with reference to FIG. 10A, output 1012 illustrates implant 1004, with arrows 1014, 1014, and 1014 representing respective forces applied to a patient's bone. Further, example bone failure 1018 is illustrated, in which bone greater than or equal to 50% of the tibial bone yield strain is represented.

FIG. 10B illustrates example graphs 1020A, 1020B representing risk of bone failure, in accordance with an example implementation of the present disclosure; In connection with cemented and uncemented fixation, patients with lower BMD tend to have more bone at risk of failure underneath the tibial baseplate. In this way, patients with cemented fixation, which generally have lower density, show increased amount of bone at risk of failure. For example, data from a large series of FEA of patients showing the amount of bone at risk of failure in patients that received cemented and uncemented implants, e.g., from a retrospective study. The choice of fixation was determined by the surgeon, not by any analysis. The data needs context as even though the analysis is labeled ‘cemented’ and ‘uncemented’ the analysis was done assuming uncemented fixation to show that the surgeons are somewhat identifying patients at risk of failure and therefore using cemented fixation in those patients i.e. the patients that had cemented fixation had a higher risk of bone failure if they had been uncemented (data shown on the right hand side).

FIG. 10C illustrates risk of failure, in connection with low bone mineral density and high bone mineral density. As shown in FIGS. 10A-10C, the present disclosure addresses technical challenges associated with identifying factors that impact implant fixation, such as tibial implant fixation. As represented in FIGS. 10A-10C, patients with a low BMD have higher risks of bone failure following arthroplasty. A non-linear relationship observed between BMD and bone failure can indicate a threshold, which is usable to identify patients that can benefit from one type of fixation 1022B (e.g., cementless) over another 1022A (e.g., cemented).

Moreover, the workflow is applicable in the field of biomechanics of total knee arthroplasty for a wide variety of clinically relevant concerns related to how patient, surgical, and implant factors affect the function and longevity of total knee arthroplasty.

Although the present disclosure is described by way of example herein in terms of a web-based system using web browsers, custom applications and a web site server (data processing apparatus 102), and with mobile computing devices, system 100 is not limited to that particular configuration. It is contemplated that system 100 can be arranged such that computing device 104 can communicate with, and display data received from, data processing apparatus 102 using any known communication and display method, for example, using a non-Internet browser Windows viewer coupled with a local area network protocol such as the Internetwork Packet Exchange (IPX). It is further contemplated that any suitable operating system can be used on computing device 104, for example, WINDOWS, MAC OS, OSX, LINUX, IOS, ANDROID and any suitable PDA or other computer operating system.

As used herein, the terms “function” or “module” refer to hardware, firmware, or software in combination with hardware and/or firmware for implementing features described herein. In the hardware sense, a module can be a functional hardware unit designed for use with other components or modules. For example, a module may be implemented using discrete electronic components, or it can form a portion of an entire electronic circuit such as an Application Specific Integrated Circuit (ASIC). Numerous other possibilities exist, and those of ordinary skill in the art will appreciate that the system can also be implemented as a combination of hardware and software modules. In the software sense, a module may be implemented as logic executing in a collection of software instructions, possibly having entry and exit points, written in a programming language, such as, for example, Java, Lua, C or C++. A software module may be compiled and linked into an executable program, installed in a dynamic link library, or may be written in an interpreted programming language such as, for example, Perl, or Python. It will be appreciated that software modules may be callable from other modules or from themselves, and/or may be invoked in response to detected events or interrupts. Software instructions may be embedded in firmware. Moreover, the modules described herein can be implemented as software modules, but may be represented in hardware or firmware. Generally, the modules described herein refer to logical modules that may be combined with other modules or divided into sub-modules despite their physical organization or storage.

While operations shown and described herein may be in a particular order, this should not be understood as requiring that such operations be performed in the particular order shown or in sequential order, or that all illustrated operations be performed, to achieve desirable results. In certain circumstances, multitasking and parallel processing can be advantageous. Moreover, the separation of various system components in the embodiments described above should not be understood as requiring such separation in all embodiments, and it should be understood that the described program components and systems can generally be integrated together in a single software product or packaged into multiple software products.

The terminology used herein is for the purpose of describing particular embodiments only and is not intended to be limiting of the invention. As used herein, the singular forms “a”, “an” and “the” are intended to include the plural forms as well, unless the context clearly indicates otherwise. It will be further understood that the terms “comprises” and/or “comprising”, when used in this disclosure, specify the presence of stated features, integers, steps, operations, elements, and/or components, but do not preclude the presence or addition of one or more other features, integers, steps, operations, elements, components, and/or groups thereof.

It should be noted that use of ordinal terms such as “first,” “second,” “third,” etc., in the claims to modify a claim element does not by itself connote any priority, precedence, or order of one claim element over another or the temporal order in which acts of a method are performed, but are used merely as labels to distinguish one claim element having a certain name from another element having a same name (but for use of the ordinal term) to distinguish the claim elements.

Also, the phraseology and terminology used herein is for the purpose of description and should not be regarded as limiting. The use of “including,” “comprising,” or “having,” “containing,” “involving,” and variations thereof herein, is meant to encompass the items listed thereafter and equivalents thereof as well as additional items.

Particular embodiments of the subject matter described in this disclosure have been described. Other embodiments are within the scope of the following claims. For example, the actions recited in the claims can be performed in a different order and still achieve desirable results. As one example, the processes depicted in the accompanying figures do not necessarily require the particular order shown, or sequential order, to achieve desirable results. In certain implementations, multitasking and parallel processing can be advantageous.

What is claimed:

1. A computerized method, comprising:
identifying at least one of an implant type and an implant fixation procedure, by at least one computing device processing surgical plan information representing details associated with an arthroplasty procedure for a patient;
determining, by the at least one computing device processing at least one image of the patient, bone mineral density information associated with the patient;
selecting, by the at least one computing device processing the determined bone mineral density information and by the at least one computing device processing patient information representing at least demographics associated with the patient, a plurality of patients that previously underwent arthroplasty including at least some of the details associated with the arthroplasty procedure for the patient;
establishing, by the at least one computing device processing outcome information representing results of arthroplasty procedures respectively associated with the plurality of patients, a threshold representing a biomechanical tolerance of the implant fixation;
determining, by the at least one computing device processing information associated with at least the determined bone mineral density information, that biomechanics of implant fixation associated with the arthroplasty procedure for the patient is above or below the established threshold; and
providing, by the at least one computing device, information associated with the determination that the bone mineral density information associated with the patient is above or below the established threshold.

2. The method of claim 1, wherein the outcome information further represents arthroplasty procedures that succeeded and arthroplasty procedures that failed due to mechanical reasons.

3. The method of claim 1, wherein establishing the threshold further includes the at least one computing device processing implant and fixation information representing implants, fixation and implant migration associated with the arthroplasty procedures respectively associated with the plurality of patients.

4. The method of claim 1, further comprising comparing, by the at least one computing device the determined bone mineral density information with information representing bone mineral densities of a plurality of patients that previously underwent knee arthroplasty.

5. The method of claim 1, wherein the bone mineral density information of the plurality of patients is displayed graphically according to a range of confidence intervals.

6. The method of claim 5, wherein the graphical representation includes a second range of confidence intervals.

7. The method of claim 5, wherein the graphical representation includes at least one patient overlay superimposed upon the range of confidence intervals.

8. The method of claim 1, wherein the automatically provided information is at least one of an alert displayed on a screen display, an audible alert, and a vibratory alert.

9. The method of claim 1, wherein the automatically provided information includes a recommendation for at least one of alignment and fixation technique.

10. The method of claim 1, wherein the automatically provided information is instructional to configure a robotic surgical system.

11. The method of claim 1, further comprising quantifying an interaction between implant and the bone, by the at least one computing device providing the determined bone mineral density information as input to a computational model.

12. The method of claim 11, further comprising providing load information associated with biomechanics of the patient as the input to the computational model.

13. The method of claim 11, wherein outcome of the model relates to an ability of the implant to achieve and maintain fixation following the arthroplasty for the patient.

14. The method of claim 13, wherein the outcome includes information associated with at least one of:
implant motion relative to the bone;
subsidence;
bone strength;
bone strains;
bone stress;
stress shielding; and
interfacial stress.

15. The method of claim 13, wherein the outcome represents a likelihood of a failure of a bond between the implant and the bone.

16. The method of claim 1, further comprising converting, by the at least one computing device processing the at least one image of the patient, Hounsfield units to bone mineral density information.

17. The method of claim 1, wherein the at least one image of the patient is a computerized (CT) scan, magnetic resonance imaging (MRI), ultrasound, or a plurality of images with three-dimensional information.

18. A computerized system, comprising:
at least one computing device, configured by executing instructions stored on non-transitory processor readable media for:
identifying at least one of an implant type and an implant fixation procedure, by processing surgical plan information representing details associated with an arthroplasty procedure for a patient;
determining, by processing at least one image of the patient, bone mineral density information associated with the patient;
selecting, by processing the determined bone mineral density information and by processing patient information representing at least demographics associated with the patient, a plurality of patients that previously underwent arthroplasty including at least some of the details associated with the arthroplasty procedure for the patient;
establishing, by processing outcome information representing results of arthroplasty procedures respectively associated with the plurality of patients, a threshold representing a biomechanical tolerance of the implant fixation;
determining, by processing information associated with at least the determined bone mineral density information, that biomechanics of implant fixation associated with the arthroplasty procedure for the patient is above or below the established threshold; and
providing information associated with the determination that the bone mineral density information associated with the patient is above or below the established threshold.

19. The system of claim 18, wherein the outcome information further represents arthroplasty procedures that succeeded and arthroplasty procedures that failed due to mechanical reasons.

20. The system of claim 18, wherein establishing the threshold further includes processing implant and fixation information representing implants, fixation and implant migration associated with the arthroplasty procedures respectively associated with the plurality of patients.
